(19) United States
(12) Patent Application Publication
(10) Pub. No.: US 2025/0367268 A1
(43) Pub. Date: Dec. 4, 2025

(54) Compositions and Methods for Treating Internal Bleeding and Hemorrhage

(71) Applicant: The Government of the United States, as Represented by the Director of the Defense Health Agency, Fort Detrick, MD (US)

(72) Inventor: Alexander Hayes Penn, San Antonio, TX (US)

(21) Appl. No.: 19/211,794

(22) Filed: May 19, 2025

Related U.S. Application Data
(60) Provisional application No. 63/654,410, filed on May 31, 2024.

(51) Int. Cl. A61K 38/38; A61K 9/00; A61K 33/00; A61K 33/14; A61K 38/42; A61P 7/08

(52) U.S. Cl. CPC A61K 38/385; A61K 9/0026; A61K 33/00; A61K 33/14; A61K 38/42; A61P 7/08

(57) ABSTRACT

Disclosed herein are resuscitation fluids and compositions comprising NEFA free blood serum albumin and hemoglobulin (cell free or as lysed red blood cells) and methods of treating internal bleeding and hemorrhage.

### CROSS-REFERENCE TO RELATED APPLICATIONS

This application claims the benefit of U.S. Patent Application No. 63/654,410, filed May 31, 2024, which is herein incorporated by reference in its entirety.

### ACKNOWLEDGEMENT OF GOVERNMENT SUPPORT

This invention was made by employees of the United States Air Force. The Government has certain rights in the invention.

### BACKGROUND OF THE INVENTION

### 1. Field of the Invention

The field generally relates to resuscitation fluids for, e.g., treating internal bleeding and hemorrhage.

### 2. Description of the Related Art

Severe trauma (i.e., internal bleeding) and hemorrhage present many challenges, with mortality rising sharply the longer care is required in the field. After hemorrhage and severe internal bleeding, there is not enough blood volume to provide the requisite pressure on the vasculature that is necessary for venous return/pre-load. The sympathetic nervous system (SNS) compensates by increasing the constriction of the vasculature to reduce the volume mis-match. Even after hemorrhage is controlled, there is a high risk of death due to decompensation, which is the failure of the vasculature to maintain sufficient constriction for blood flow to vital organs. Decompensation may occur slowly, over hours, or acutely, within minutes.

Resuscitation fluids are administered, usually intravenously, to prevent and treat decompensation. Such intravenous fluids include whole blood, plasma, colloids (e.g., albumin, starches, dextrans, gelatins, etc.), and crystalloids (salt and electrolyte solutions). Resuscitation fluids containing albumin administered after hemorrhagic shock have been shown to reduce both mortality and fluid volume replacement requirements. Unfortunately, due to the presence of non-esterified fatty acids (NEFA), the current albumin formulation causes undesirable vascular permeability/leakage, which may result in complications and worsen injuries. In fact, compared to saline, albumin made traumatic brain injuries worse by increasing intracranial pressures.

### SUMMARY OF THE INVENTION

Provided herein are compositions comprising, consisting essentially of, or consisting of a blood serum albumin and a hemoglobulin and a total non-esterified fatty acid (NEFA) content of 0.14 wt % or less, preferably 0.07 wt % or less, of the total weight of the blood serum albumin. In some embodiments, the blood serum albumin, the hemoglobulin, and the total NEFA content are present in a ratio, based on weight, of 40.0-260.0:7.2-100.0:0.0-0.5, preferably 50.0-250.0:9.0-96.0:0.0-0.4, respectively. In some embodiments, the hemoglobulin is provided in the form of lysed red blood cells. In some embodiments, the amount of lysed red blood cells is about 1.0×10⁹-5.9×10¹⁰ per 1000 grams of the blood serum albumin. In some embodiments, the compositions further comprise, per 1000 parts by weight of the blood serum albumin, one or more of the following: Sodium Chloride 20.0-110.5 parts; Sodium Gluconate 19.0-105.4 parts; Sodium Acetate 14.0-77.3 parts; Potassium Chloride 1.4-7.8 parts; and/or Magnesium Chloride 1.1-6.3 parts. In some embodiments, the blood serum albumin is human serum albumin. In some embodiments, the hemoglobulin is human hemoglobulin.

Provided herein are methods of treating a subject for internal bleeding and/or hemorrhage, which comprise intravenously administering to the subject a blood serum albumin, a hemoglobulin, and, if administered, an amount of non-esterified fatty acids (NEFAs) that is not more than 0.14 wt %, preferably not more than 0.07 wt %, of the administered amount of the blood serum albumin. In some embodiments, the blood serum albumin and the hemoglobulin are administered concurrently or sequentially. In some embodiments, the blood serum albumin and the hemoglobulin are administered in the form of a composition which comprises, consists essentially of, or consists of a blood serum albumin and a hemoglobulin and a total NEFA content of 0.14 wt % or less, preferably 0.07 wt % or less, of the total weight of the blood serum albumin in the composition. In some embodiments, the blood serum albumin, the hemoglobulin, and the total NEFA content are present in the composition at a ratio, based on weight, of 40.0-260.0:7.2-100.0:0.0-0.5, preferably 50.0-250.0:9.0-96.0:0.0-0.4, respectively. In some embodiments, the hemoglobulin is provided in the form of lysed red blood cells. In some embodiments, the amount of lysed red blood cells is about 1.0×10⁹-5.9×10¹⁰ per 1000 grams of the blood serum albumin administered. In some embodiments, the methods further comprise administering to the subject sodium chloride, sodium gluconate, sodium acetate, potassium chloride, and/or magnesium chloride. In some embodiments, the lysed red blood cells are prepared by mixing a volume of whole blood with a volume of sterile water at a ratio of about 1:3 to 1:1. In some embodiments, the lysed red blood cells are prepared by mixing a volume of concentrated red blood cells with a volume of sterile water at a ratio of about 2:3 to 1:1. In some embodiments, the blood serum albumin is human serum albumin. In some embodiments, the hemoglobulin is human hemoglobulin.

Also provided herein are kits comprising (a) a blood serum albumin containing 0.14 wt % or less, preferably 0.07 wt % or less, of NEFAs, and (b) a crystalloid, a hemoglobulin, and/or lysed red blood cells packaged together. In some embodiments, the blood serum albumin is human serum albumin. In some embodiments, the hemoglobulin is human hemoglobulin.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### Additional Embodiments

Embodiment 1: A composition which comprises, consists essentially of, or consists of a blood serum albumin and a hemoglobulin and a total non-esterified fatty acid (NEFA) content of 0.14 wt % or less, preferably 0.07 wt % or less, of the total weight of the blood serum albumin.

Embodiment 2: The composition according to Embodiment 1, wherein the blood serum albumin, the hemoglobulin, and the total NEFA content are present in a ratio, based on weight, of 40.0-260.0:7.2-100.0:0.0-0.5, preferably 50.0-250.0:9.0-96.0:0.0-0.4, respectively.

Embodiment 3: The composition according to Embodiment 1 or Embodiment 2, wherein the hemoglobulin is provided in the form of lysed red blood cells.

Embodiment 4: The composition according to Embodiment 3, wherein the amount of lysed red blood cells is about 1.0×10⁹-5.9×10¹⁰ per 1000 grams of the blood serum albumin.

Embodiment 5: The composition according to any one of Embodiments 1-4, further comprising, per 1000 parts by weight of the blood serum albumin, one or more of the following: Sodium Chloride 20.0-110.5 parts; Sodium Gluconate 19.0-105.4 parts; Sodium Acetate 14.0-77.3 parts; Potassium Chloride 1.4-7.8 parts; and/or Magnesium Chloride 1.1-6.3 parts.

Embodiment 6: A method of treating a subject for internal bleeding and/or hemorrhage, which comprises intravenously administering to the subject a blood serum albumin, a hemoglobulin, and, if administered, an amount of non-esterified fatty acids (NEFAs) that is not more than 0.14 wt %, preferably not more than 0.07 wt %, of the administered amount of the blood serum albumin.

Embodiment 7: The method according to Embodiment 6, wherein the blood serum albumin and the hemoglobulin are administered concurrently or sequentially.

Embodiment 8: The method according to Embodiment 6 or Embodiment 7, wherein the blood serum albumin and the hemoglobulin are administered in the form of a composition which comprises, consists essentially of, or consists of a blood serum albumin and a hemoglobulin and a total NEFA content of 0.14 wt % or less, preferably 0.07 wt % or less, of the total weight of the blood serum albumin in the composition.

Embodiment 9: The method according to Embodiment 8, wherein the blood serum albumin, the hemoglobulin, and the total NEFA content are present in the composition at a ratio, based on weight, of 40.0-260.0:7.2-100.0:0.0-0.5, preferably 50.0-250.0:9.0-96.0:0.0-0.4, respectively.

Embodiment 10: The method according to Embodiment 8 or Embodiment 9, wherein the hemoglobulin is provided in the form of lysed red blood cells.

Embodiment 11: The method according to Embodiment 10, wherein the amount of lysed red blood cells is about 1.0×10⁹-5.9×10¹⁰ per 1000 grams of the blood serum albumin administered.

Embodiment 12: The method according to any one of Embodiments 6-11, which further comprises administering to the subject sodium chloride, sodium gluconate, sodium acetate, potassium chloride, and/or magnesium chloride.

Embodiment 13: The method according to any one of Embodiments 6-12, wherein the lysed red blood cells are prepared by mixing a volume of whole blood with a volume of sterile water at a ratio of about 1:3 to 1:1.

Embodiment 14: The method according to any one of Embodiments 6-12, wherein the lysed red blood cells are prepared by mixing a volume of concentrated red blood cells with a volume of sterile water at a ratio of about 2:3 to 1:1.

Embodiment 15: A kit comprising (a) a blood serum albumin containing 0.14 wt % or less, preferably 0.07 wt % or less, of NEFAs, and (b) a crystalloid, a hemoglobulin, and/or lysed red blood cells packaged together.

Embodiment 16: The composition according to any one of Embodiments 1-5, the method according to any one of Embodiments 6-14, or the kit according to Embodiment 15, wherein the blood serum albumin is human serum albumin.

Embodiment 17: The composition according to any one of Embodiments 1-5, the method according to any one of Embodiments 6-14, or the kit according to Embodiment 15, wherein the hemoglobulin is human hemoglobulin.

### DESCRIPTION OF THE DRAWINGS

This invention is further understood by reference to the drawings wherein:

FIG. 1, FIG. 2, and FIG. 3 provide the experimental results evidencing that NEFAs cause vascular leakage. FIG. 1 shows that bovine albumin with oleic acid (“OA-sat. BSA”) or albumin that is NEFA-free albumin (“FA-free BSA”) added immediately after T1 (the end of shock) initially raises the plasma protein level measured at T2 (1 hour after T1). However, by T4 (3 hours after T1) the protein levels drop in the OA-BSA group. This drop does not occur in the NEFA-free Albumin Group or the Crystalloid Only Group (“Plasmalyte”, control group). FIG. 2 shows that same significant drop occurs regardless of if treatment is with BSA saturated with oleic acid (OA) or BSA saturated with caprylic acid (CA) or pharmaceutical human albumin (HSA) (and that the drop is significant for the pharmaceutical albumin). FIG. 3 shows that, combined, the NEFA-containing albumins cause significant protein loss, but the crystalloid control does not.

FIG. 4 is a graph showing that NEFA-containing albumin improves survival at 3 hours after hemorrhage compared to crystalloid (“Plasmalyte”) alone.

FIG. 5 to FIG. 11 provide results from “Experiment 1” (30 ml/kg hemorrhage and isoflurane at 1% during the shock period) comparing Crystalloid (“Plasmalyte”) vs. Hemolysate resuscitation. Survival (FIG. 5), plasma lactate (FIG. 6), resuscitation volume (FIG. 7), estimated blood volume (FIG. 8), mean arterial pressure (FIG. 9), diastolic arterial pressure (FIG. 10), and arterial pulse pressure (FIG. 11). N (the number of animals contributing to the data shown) started at 10 in both groups and decreases with time as animals succumb to shock as shown in FIG. 5. FIG. 7 and FIG. 8 can be converted to ml/kg through the relation 100%=60 ml/kg. FIG. 9, FIG. 10, and FIG. 11 include all time points prior to the onset of acute decompensation. Data expressed as mean±SD. *p<0.05. NS=not significant.

FIG. 12 to FIG. 18 provide results from “Experiment 2” (30 ml/kg hemorrhage and isoflurane at 1% during the shock period) comparing NEFA-free albumin+crystalloid (“BSA+Plasmalyte”) vs. NEFA-free albumin+Hemolysate (“BSA+Hemolysate”) resuscitation. Survival (FIG. 12), plasma lactate (FIG. 13), resuscitation volume (FIG. 14), estimated blood volume (FIG. 15), mean arterial pressure (FIG. 16), diastolic arterial pressure (FIG. 17), and arterial pulse pressure (FIG. 18). N (the number of animals contributing to the data shown) started at 10 in both groups and decreases with time as animals succumb to shock as shown in FIG. 12. FIG. 15 and FIG. 14 can be converted to ml/kg through the relation 100%=60 ml/kg. FIG. 16, FIG. 17, and FIG. 18 include all time points prior to the onset of acute decompensation. Data expressed as mean±SD. *p<0.05. NS=not significant.

FIG. 19 to FIG. 27 provide results from “Experiment 3” and “Experiment 4” comparing NEFA-free albumin+crystalloid (“BSA+Plasmalyte”) vs. NEFA-free albumin+Hemolysate (“BSA+Hemolysate”) resuscitation. Experiment 3 is the lowest severity model (27 ml/kg hemorrhage and 1.5% isoflurane during the shock period). Experiment 4 is the intermediate severity model (between Experiment 4 and Experiments 1 and 2) (27 ml/kg hemorrhage and 2% isoflurane during the shock period). Plasma lactate in the Experiment 3 (FIG. 19) and Experiment 4 (FIG. 20). Survival (FIG. 21) and mean arterial pressure (FIG. 22) in Experiment 3. Plasma hemoglobin in Experiment 3 (FIG. 23) and Experiment 4 (FIG. 24). N (the number of animals contributing to the data shown) is shown at the base of each bar. Survival (FIG. 25), time that mean arterial pressure remained responsive to fluid (i.e., time to shock irreversibility) (FIG. 26), and mean arterial pressure (FIG. 27) in Experiment 4. Data expressed as mean±SD or in Kaplan-Meier curves (for survival and time to shock irreversibility). *p<0.05. BSA=bovine serum albumin. iso=isoflurane. Exp.=Experiment.

FIG. 28 is a schematic of experimental timeline. MAP=mean arterial pressure. PFC=prolonged field care. Plasmalyte=crystalloid.

### DETAILED DESCRIPTION OF THE INVENTION

Pharmaceutical grade albumin comes highly saturated with non-esterified fatty acids (NEFAs) and is used in resuscitation fluids to treat hemorrhage and/or treat or prevent decompensation by restoring plasma volume. Resuscitation fluids containing albumin and NEFAs cause vascular leakage (i.e., increased vascular permeability) and thereby worsen some injuries, e.g., increase intracranial pressures in traumatic brain injuries.

As disclosed herein, the NEFAs from albumin are the cause of the vascular leakage. It is believed that the NEFAs damage the endothelial cells of blood vessels and thereby cause vascular leakage. While NEFA-free albumin does not cause vascular leakage, it fails to provide the same therapeutic benefits as NEFA-containing albumin in treating and preventing decompensation after severe hemorrhage. NEFA from albumin damage and lyse red blood cells (RBCs). It is believed the damaged RBCs help raise blood pressure and prevent decompensation, via the release or increased proximity to the vascular walls of hemoglobin (Hb), a nitric oxide scavenger. Since nitric oxide is a vasodilator, the net effect is one of vasoconstriction. The experiments herein also show that hemolysate (i.e., damaged and/or lysed red blood cells suspended in crystalloid) increases blood pressure and reduces fluid requirements after hemorrhage, but tends to worsen ischemia and has a reduced ability to inhibit acute decompensation from severe hemorrhage (50% bleed).

NEFA-free albumin combined with hemolysate, however, raises blood pressure and reduces fluid requirements better than crystalloid alone, hemolysate alone, and NEFA-free albumin with or without crystalloid. The combination of NEFA-free albumin and hemolysate also reduces the severity and incidence of ischemia and mortality compared to treatment with hemolysate alone.

Thus, provided herein are compositions that provide the therapeutic benefits of albumins that contain NEFAs but without the undesired vascular leakage. The compositions comprise, consist essentially of, or consist of (a) NEFA-free albumin, (b) hemoglobulin, preferably provided in the form of lysed red blood cells, c) a crystalloid, and (d) a total NEFA content of 0-0.14%, preferably 0-0.07%, more preferably 0-0.05%, and most preferably 0-0.02%, by weight of the total amount of albumin.

As used herein, “NEFA-free albumin” refers to an albumin preparation that contains a NEFA content of 0-0.14%, preferably 0-0.07%, more preferably 0-0.05%, and most preferably 0-0.02% by weight of albumin. The albumin may be recombinantly produced albumin or albumin purified from blood from, e.g., bovine or humans, and treated using methods in the art to remove or reduce the amount of NEFAs.

### Albumin Studies

Two studies of resuscitation by albumin after hemorrhage were performed. In the first study, resuscitation by a crystalloid (control) was compared to resuscitation with either a bolus of bovine serum albumin (BSA) saturated with oleic acid (OA-BSA) or a bolus of NEFA-free albumin, followed by crystalloid, as needed. As commercially available pharmaceutical grade albumin is human albumin (HSA) that is nearly saturated with caprylic acid, instead of oleic acid, in the second study OA-BSA, caprylic acid-saturated BSA (CA-BSA), and pharmaceutical grade human serum albumin (HSA) were compared. A crystalloid-only group (“Crystalloid Only Group”) was also run as a control. Note that HSA is usually 1.2% caprylic acid by weight of albumin plus an additional approximate 0.14% by weight of albumin of assorted NEFAs originating from the donor, for a total of approximately 1.34% NEFA by weight of albumin. In the second study, the consistent significant difference in hemodynamics found among the Albumin Groups was in systolic arterial pressure (SAP) between the CA-BSA and HSA groups that began immediately after bolus infusion and lasted approximately 30 minutes and was between 10 and 15 mmHg in magnitude. There were no significant differences among the Albumin Groups (all containing high concentrations of NEFA) in any of the other measurements.

### Results

### Permeability

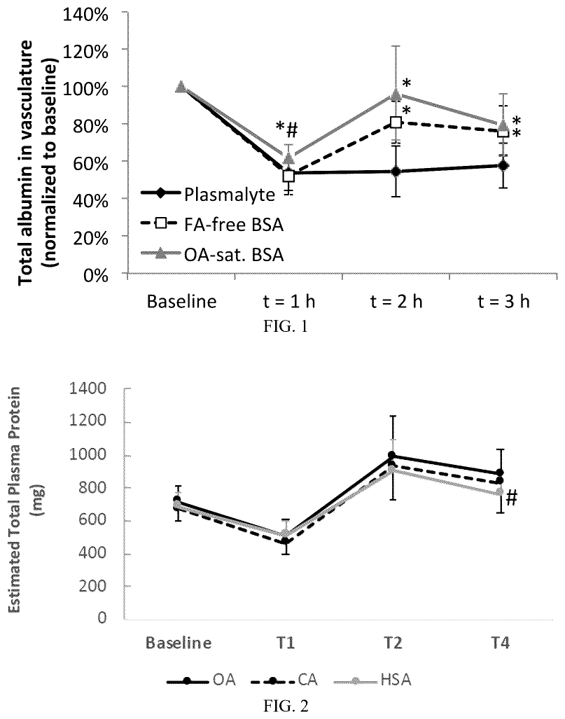

In the first study, after treatment, protein (particularly albumin) was lost from the circulation in the subjects of the OA-BSA group, but not in the subjects of the NEFA-free albumin and control groups (FIG. 1). These results indicate that the NEFAs (i.e., oleic acid and caprylic acid) cause vascular permeability to proteins to increase. In the second study, all three of the NEFA-containing albumins, but not the Crystalloid Only Group, lost protein from the circulation. The loss of protein was significant for the HSA group and the groups treated with NEFA-containing albumins as a whole (FIG. 2 and FIG. 3).

### Mortality

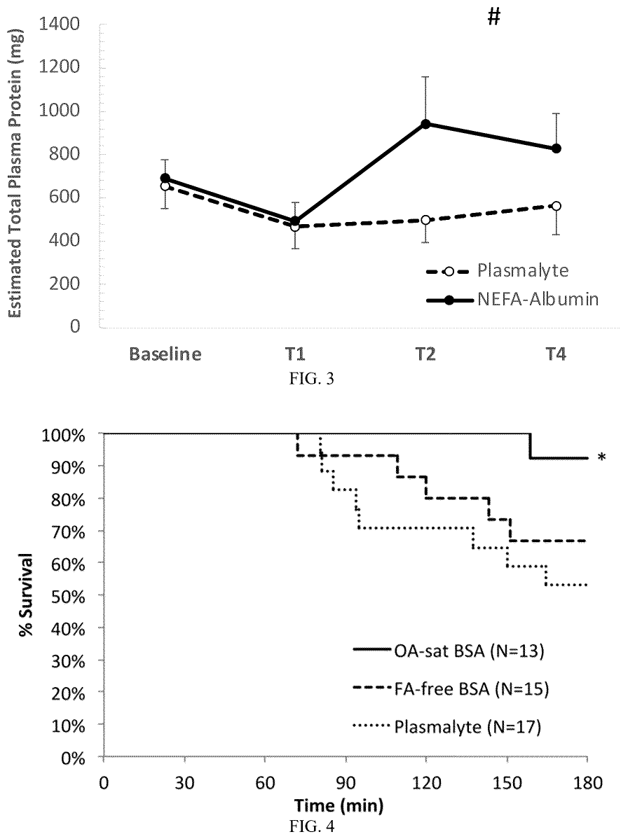

In the first study, OA-BSA, but not NEFA-free albumin, significantly reduced mortality compared to the Crystalloid Only Group (reduced from 48% to 8%) (FIG. 4). Unfortunately, in the second study, due to some changes to the anesthesia regimen used in the model, there was too little mortality in the control group (2 of 15) to determine if the NEFA-containing albumins would have improved survival. The near identical effects of the three NEFA-containing albumins in all other measures, combined with finding of improved survival with OA-BSA in the first study, however, suggest HSA would likely have also improved survival compared to crystalloid alone.

### Hemodynamics

In both studies, the bolus of NEFA-containing albumin created a fast, sharp increase in mean arterial pressure (MAP), resulting mostly from a rise in diastolic arterial pressure (DAP), but also a small increase in pulse pressure (PP). This increase in pressure was well above the threshold pressure that would have triggered us to give additional crystalloid. While the MAP slowly dropped from its peak of ˜85 mmHg over the first hour, it generally settled at or just above the transfusion trigger pressure of 50 mmHg. In contrast, the Crystalloid Only Group exceeded the trigger pressure only briefly, if at all, resulting in a higher fluid infusion rate. NEFA-free albumin acted on pressure in a fashion intermediate between the other two groups. The pressure difference between OA-BSA and NEFA-free albumin indicates that OA-BSA causes vasoconstriction in addition to the effect 25% albumin has of pulling fluid into the vasculature via oncotic pressure. This is understood because NEFA has extremely low solubility in water, which is why it either stays bound to albumin or inserts into cell membranes, and thus its effect on pressure is more likely due to vasoconstriction than to osmotic or oncotic pressure increases.

In the second study, the NEFA-containing albumins were similarly able to strongly increase blood pressure during the first hour after infusion, with a lingering improvement thereafter relative to the crystalloid control.

### Fluid Requirements

In the first study, since MAP stayed above the resuscitation threshold in most cases in the OA-BSA group, the amount of resuscitation fluid required to keep pressure at a sufficient level was significantly reduced from 56 ml/kg in the control group to 6 ml/kg with OA-BSA. The NEFA-Free Group, in contrast, needed 46 ml/kg. While the NEFA-Free Group needed relatively little fluid in the first hour of resuscitation, it needed approximately as much fluid as the control group in the second hour.

In the second study, since MAP stayed above the resuscitation threshold in nearly all cases in the Albumin Group, the median resuscitation (and upper quartile) in the first hour was equal to the 2.95 ml/kg bolus itself. Likewise, the median resuscitations in the second and third hours of resuscitation were zero in the Albumin Group. In contrast, the median animal in the Crystalloid Only Group needed a fairly constant 9 ml/kg per hour. The median total fluid needed over the three hours was 3.75 (interquartile range (IQR): 2.95-6.64) ml/kg in the Albumin Groups, only 0.8 ml/kg more than the bolus itself. The Crystalloid Only Group needed more than 10 times as much total volume (39.36 (IQR: 18.38-44.91) ml/kg; p<0.000001). Hematocrit was identical between groups through the end of shock, but after treatment dropped more in the Albumin Groups than the Crystalloid Only Group. This information was used to estimate blood volumes. Hemorrhage was designed to remove 45% of baseline blood volume, but by T1, plasma volume recovered from 55% to 90% of baseline due to “auto-resuscitation”, bringing the total blood volume to 75% of baseline by T1. Treatment with albumin further increased the total blood volume to 93% at T2, though this decreased slightly to 87% at T4 (a blood volume increase from T1 to T4 of 7.1 ml/kg). In comparison, crystalloid alone increased volume to 79% of baseline by T4 (a blood volume increase of 3.7 ml/kg from T1 to T4). Comparing this to the volume administered, it is estimated that less than a tenth of the fluid given in the Crystalloid Only Group remained in circulation, whereas albumin pulled ˜3.4 ml/kg of extravascular fluid into the vasculature.

Other Measures from the Second Study

Lactate was identical between groups at baseline and at the end of shock, however within an hour of starting resuscitation lactate returned to near baseline levels in the Albumin Groups, whereas the Crystalloid Only Group remained significantly (p<0.004) elevated versus the Albumin Groups at T2 and T4. Femoral venous oxygen saturation (SvO2) dropped from ˜56% at baseline to ˜23% at the end of shock in all groups. After resuscitation, SvO2 increased significantly (p=0.00002) more in the Albumin Groups than it did in the Crystalloid Only Group (45% versus 30% at T2), but by T4, both groups had venous oxygen saturation (SvO2) of ˜35%. Interestingly, plasma glucose concentration for all groups increased from baseline to T1 and began dropping again after resuscitation to a similar value at T2. However, while the Albumin Group glucose dropped to slightly below baseline by T4, the drop was significantly (p=0.008) greater in the Crystalloid Only Group at T4, despite the lower plasma expansion in that group. Hemorrhage presumably removed 45% of total protein from circulation, though by T1 total protein was 71% of baseline (as compared to plasma volume, which increased to 90% of baseline), reflecting the fact that auto-resuscitation fluid has some protein (though less than plasma). As expected, protein increased after the albumin bolus and remained steady in the Crystalloid Only Group. However, while total protein changed little in the Crystalloid Only Group from T2 to T4, it dropped significantly (p=0.001) in the Albumin Groups, suggesting the permeability of the vasculature to large molecules increased.

There were no observable differences in BALF cfHb (i.e., cell-free hemoglobulin in bronchoalveolar lavage fluid (BALF)) between groups. However, BALF cfHb correlated strongly with BALF albumin (i.e., albumin in BALF) (correlation coefficient=0.995, p<0.0001) and with lung perivascular edema histology score (correlation coefficient=0.593, p<0.0001). There was also no observable difference between groups in lung moisture content (71.0±6.8% for crystalloid and 72.2±4.7% for albumin). No clinically significant histological findings were observed in the heart, kidney, intestine, spleen, or liver.

### Discussion (Second Study)

### Hemodynamics

Over the course of the first 5-10 minutes after administration, the bolus of albumin caused diastolic pressure to nearly double and increased pulse pressure by about 15-20%. Based on the findings in the first study, approximately half of the increase in diastolic arterial pressure (DAP) is due to the effect of albumin (i.e., influx of fluid into the intravascular space due to increased oncotic pressure) and half from the effect of the NEFA. The increase in pulse pressure (PP) in the Albumin Groups is most likely from the plasma expansion reducing blood viscosity, and therefore systemic resistance, which in turn increases venous return and stroke volume. The increase in DAP diminished with time but did not go away completely, suggesting that there may be ongoing hemolysis adding low levels of hemoglobulin (Hb) to the circulation or that the resulting increase in vascular constriction can persist after Hb is removed from circulation. The PP increase persisted for the duration, which likely reflects the persistence of the plasma expansion.

It is believed that the persistent improvement in pressure with albumin is the result of simultaneous volume infusion and anti-dilatory action (via Hb). Therefore, the dose of albumin may be split into several doses that are administered over time to avoid peak systolic pressures that mostly disappear 15 minutes after the single administration, without losing the longer-term hemodynamic benefits.

Between T1 and T2 (i.e., 1-2 hours after hemorrhage), the Crystalloid Only Group hemodynamics remained constant, maintained by a steady infusion of fluid. Between T2 and T4 (i.e., 2-4 hours after hemorrhage) in the Crystalloid Only Group, there was a non-significant, mild decrease in DAP and simultaneous mild increase in PP that was reminiscent of the more extreme hemodynamic changes seen in the first study. These trends were not present in the Albumin Groups, suggesting that protection from decompensation is likely common to albumins and not unique to OA-saturated BSA. The transition from steady to decompensating at T2 suggests that even the small reduction in volume from the blood sample can have major effects when the subject remains hypovolemic. Alternatively, the removal of the tourniquet may have played a role, through release of mediators from the leg or a reduction in sympathetic signaling in response to reduced ischemia.

### Circulating Volume and Fluid Requirements

Adding crystalloid lowers plasma oncotic pressure. The crystalloid increases the extravasation of water at the capillaries until the plasma oncotic pressure recovers enough to match hydrostatic pressure. This explains why there was so little net gain in circulating volume in the Crystalloid Only Group, despite large volume infusion. That there was any net gain is likely because hydrostatic pressure (i.e., DAP) also dropped mildly. In the absence of sufficient circulating volume, MAP was below the resuscitation trigger pressure, setting up an endless loop of resuscitation and fluid extravasation, even in the absence of much decompensation. More complicated to explain is how little extravascular fluid was recruited into the vasculature by the albumins. Given that the albumin bolus concentration is roughly 5 times the concentration in plasma, one might think ˜12 ml/kg (4 times the bolus) of fluid should have been pulled into circulation, instead of the ˜3.4 ml/kg that was calculated. However, that fails to take into account the large auto-transfusion, prior to bolus treatment. Auto-transfusion fluid is derived from the extravascular volume and/or lymph and contains significantly less protein than plasma. This is seen by a 29% drop in plasma protein from Baseline to T1, despite only a 10% drop in plasma volume in that same time period (i.e., after hemorrhage and auto-resuscitation). One of the common concerns regarding resuscitation with hyper-oncotic albumin is that it could dehydrate the subject. However, little additional fluid movement was observed. Instead, the albumin from the bolus appeared to fortify the albumin-poor auto-resuscitation fluid already present.

Lactate is an indicator of tissue hypoxia, and, despite all the resources of the medical system, mortality in subjects in circulatory shock increases from 10 to 90% as lactate increases from 2 to 8 mM. As compared to the Crystalloid Only Group, albumins were superior at returning lactate to near baseline levels. This can be explained by albumins increased blood pressure and expanded plasma volume compared to crystalloid, both of which should help restore circulation to ischemic capillary beds. Since the oxygen carrying capacity of the blood should be identical in all groups, the initial improvement in femoral SvO2 in the Albumin Groups could represent improved blood flow rates and therefore lower capillary transit times, leading to less O2 removal. Alternatively, there may have been increased O2 consumption in the leg in the Crystalloid Only Group. The latter is plausible considering that by T4 glucose was significantly lower in the Crystalloid Only Group, suggesting a hyper-metabolic state. Alternatively, the lower glucose may be from extravasation of glucose by fluid convection and replacement by crystalloid lacking glucose.

### Circulating Protein and Vascular Lung Permeability to Protein

While there was a non-significant trend for the Crystalloid Only Group to gain protein between T2 and T4, the Albumin Groups lost a significant mass of protein from the circulation during that period. This suggests that vascular permeability to larger molecules, like proteins, after hemorrhage may be dependent on the amount of NEFA entering the circulation.

The lung circulation, however, may be a special case. The capillary bed of the lungs is the first seen by the blood after lymph is added at the thoracic duct. Hemolysis, as indicated by BALF cfHb, occurred in every group, but there were no differences between groups. An extremely high linear correlation between BALF cfHb and BALF albumin and a strong correlation between BALF cfHb and the presence of histological signs of edema and damage were found. BALF albumin is a measure of lung trans-alveolar permeability. The absence of any points with high permeability (albumin) but low hemolysis (cfHb), suggests that high permeability is always accompanied by hemolysis. Similarly, the absence of any points with low permeability (albumin) but high hemolysis (cfHb), suggests that measurement of intravascular hemolysis via BALF is limited by the extent of lung permeability and therefore is under-reported.

Thus, NEFAs appear to be responsible for high permeability in both lung and systemic vasculature, but the effect depends on the source of the NEFAs. NEFAs from the ischemic gut is at least partially unbound and cytotoxic in the lymph and may still be unbound to a large extent when it reaches the lungs, increasing the damage that occurs there. Likewise, models of acute respiratory distress syndrome usually add oleic acid without any albumin (i.e., totally unbound), or with oleic acid in concentrations far above what is bindable by the albumin present. In contrast, NEFAs in pharmaceutical albumin begins in the bound state, which may lead to a slower release, less likely to target the lungs specifically. While no group differences in lung permeability or histologic injury were observed, the loss of protein from circulation indicates that the treatment is not without cost. Damage to other tissues (e.g., red blood cells) from NEFAs can release cell debris that could become trapped in the lungs and, given more time, lead to lung injury. It is believed that the NEFAs cause permeability to increase in the blood brain barrier, and thereby worsen traumatic brain injury (TBI) as a result of increased intracranial pressure.

### Hemolysate Studies

Because mortality from decompensation was prevented by treatment with a bolus of 25% albumin saturated with oleic acid prior to resuscitation with crystalloid, compared to treatment with crystalloid or 25% NEFA-free albumin, it was hypothesized that oleic acid affected compensatory mechanisms by causing intravascular hemolysis, which releases cfHb into circulation. cfHb scavenges nitric oxide, a vasodilator. Thus, removal of this vasodilator increases blood vessel constriction, increasing the proportion of total blood volume that is stressed volume, thereby improving hemodynamics with less effort from blood vessels. It is believed that this may help delay the slow form of decompensation that comes from deterioration in the ability of vessels to respond to sympathetic signals and make constriction easier by lessen the amount of sympathetic signaling required by the brain. Experience suggests that the harder the sympathetic nervous system works, the more likely it is to stop signaling completely, which is believed to be the cause of acute decompensations that can lead to death only minutes after their onset.

Unfortunately, in vivo hemolysis releases cell debris that could be potentially harmful over longer periods and, in vivo, NEFA appears to have increased permeability of blood vessels and lung epithelium, both of which would have detrimental effects over longer periods. Therefore, further experiments were conducted to determine whether cfHb is the reason for the reduction in mortality from decompensation and whether exogenously administered cfHb or hemolysate may provide the same therapeutic benefits as that from in vivo hemolysis caused by NEFAs. Because the effect of nitric oxide reduction affects veins before arteries, it was hypothesized that the administration of cfHb and hemolysate may improve venous constriction (compensating for hypovolemia) and blood pressure without increasing arterial constriction. Four experiments were performed to evaluate the effects of hemolysate (Experiment 1) and then hemolysate combined with albumin (Experiments 2-4).

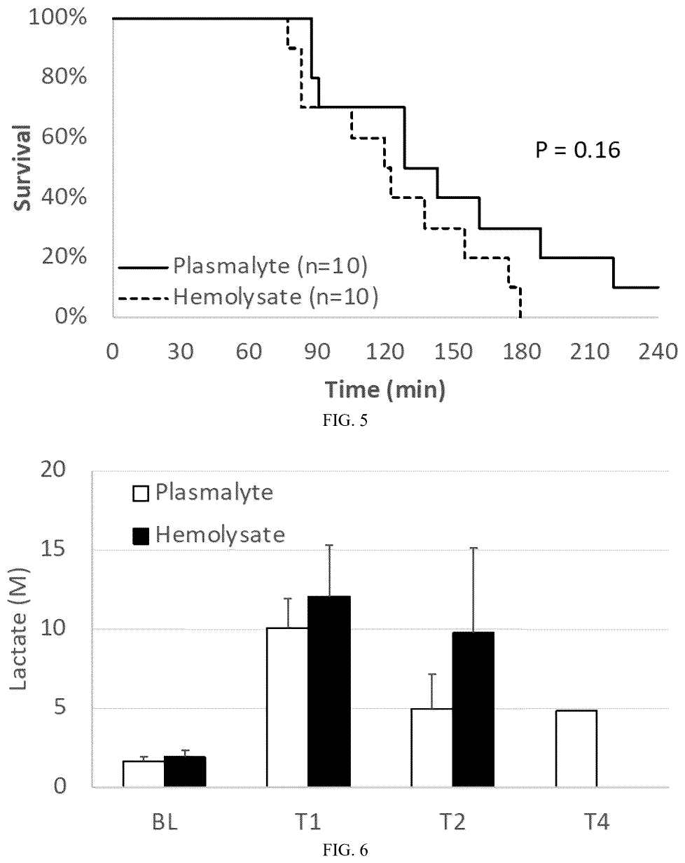

While most of the hemolysis likely occurred within minutes of bolus treatment in the albumin experiments, hemolysate was introduced more slowly in these studies. Initial experiments with a low concentration of hemolysate (1:49 Hemolysate to crystalloid) increased pressure too slowly to limit the volume of resuscitation fluid needed. Therefore, a higher concentration (1:9 Hemolysate to crystalloid) was used. The 1:9 concentration provided significantly better pressure responses to fluid, which significantly lowered the rate at which fluid was required. That is, the resuscitation target mean arterial pressure (MAP) of 60 mmHg was consistently easier to reach and maintain with significantly less fluid in the Hemolysate group (0.32±0.21 ml/min vs. 0.52±0.23 ml/min with crystalloid only, which when normalized by body weight is 0.92±0.61 ml/kg/min vs. 1.59±0.70 ml/kg/min with crystalloid only, p=0.036), and resulted in significantly lower fluid requirements at 1 h into resuscitation in Experiment 1 (T2 in FIG. 7). Unfortunately, while there was little sign of slow decompensation (e.g., no observable decrease in diastolic pressure despite constant mean arterial pressure) in the Hemolysate Group, the hemolysate did not inhibit acute decompensations. Contrary to the hypothesis, the Hemolysate Group tended to acutely decompensate earlier than the Crystalloid Only Group (FIG. 5). That is, the hemolysate treatment did not prevent or inhibit acute decompensation as hypothesized from the studies with albumin.

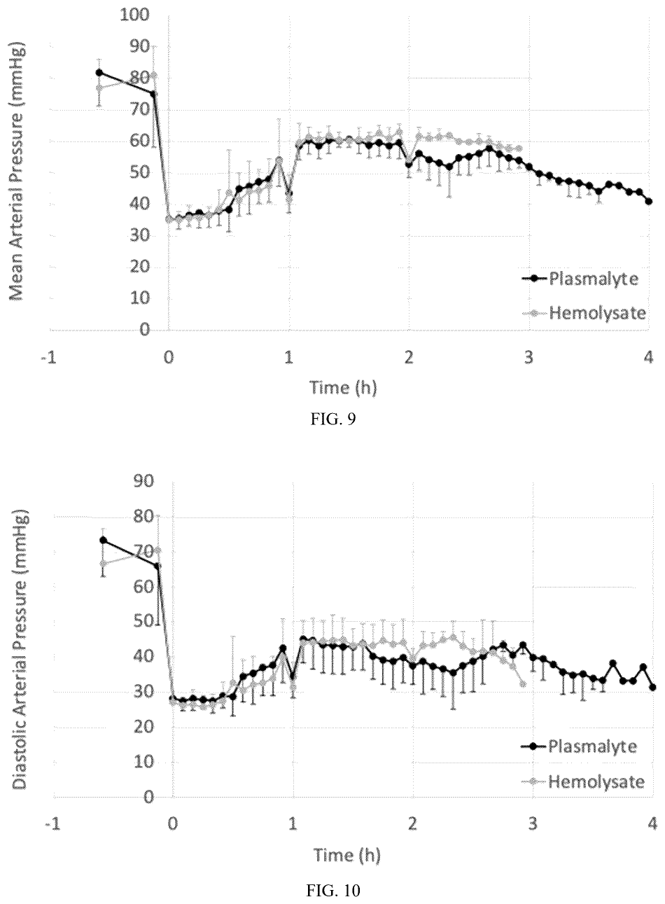

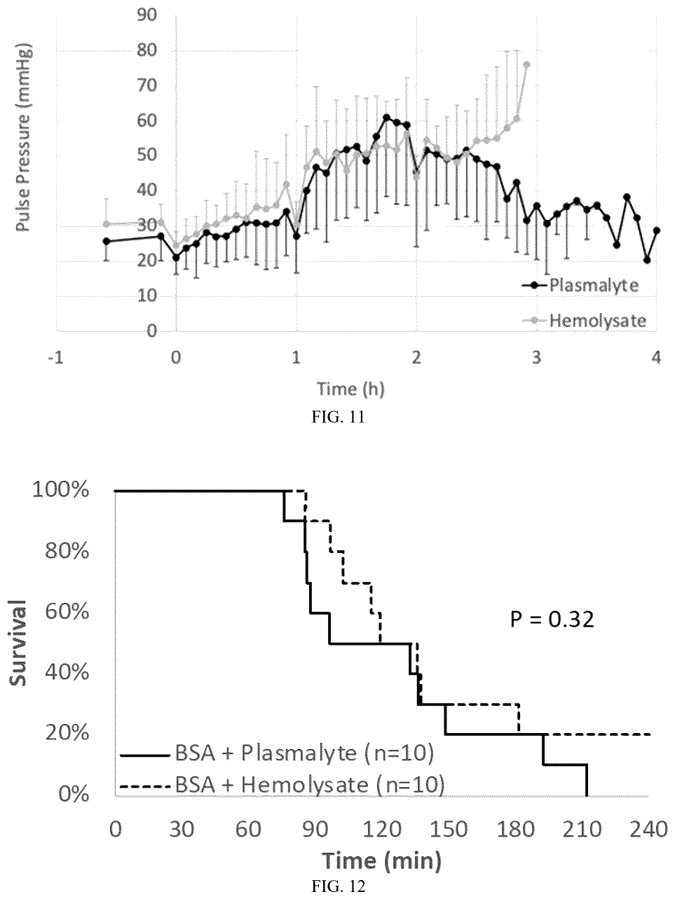

Acute decompensations are fast, usually fatal, pressure drops that can occur despite ongoing rapid fluid administration. The time between onset of acute decompensation and death over both groups was only 4 minutes on average. Blood pressure up until the start of acute decompensation gives little to no indication of an imminent crisis (FIG. 9, FIG. 10, FIG. 11). The mean arterial pressure immediately prior to each acute decompensation was 58±3 mmHg in the Hemolysate group and 51±9 mmHg in the Crystalloid Only Group (p=0.05, the Crystalloid Only Group was lower on average than the goal resuscitation pressure of 60 mmHg because 4 of the 9 non-survivors had already reached their maximum resuscitation volume (FIG. 7).

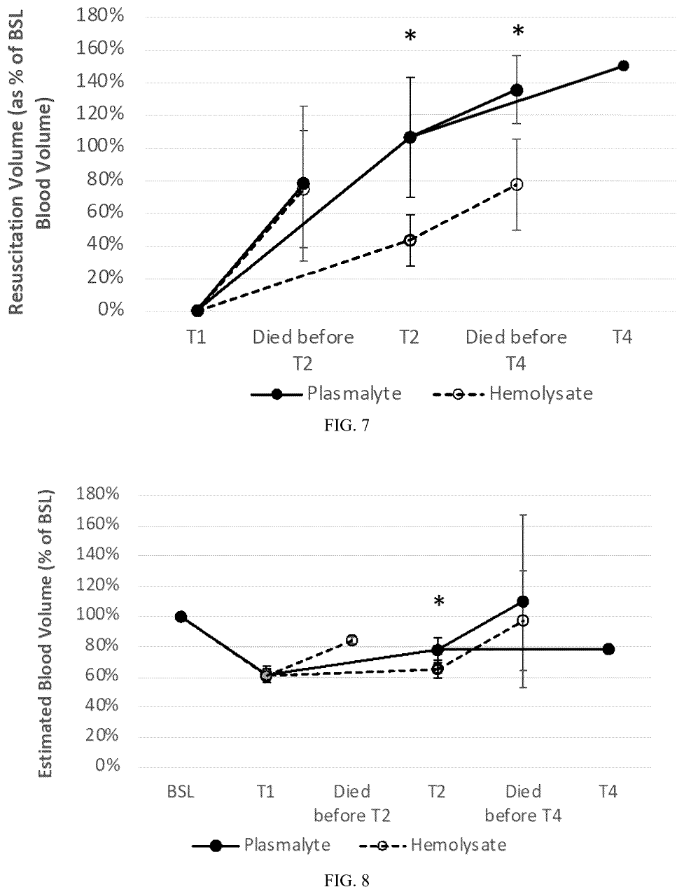

In the estimates of circulating blood volume (based on changes in hematocrit and the assumption of 60 ml/kg blood volume at baseline), there was almost no increase in blood volume between T1 and T2 in the Hemolysate Group (significantly less than the Crystalloid Only Group) despite receiving nearly as much resuscitation fluid as had been lost in hemorrhage (FIG. 7 and FIG. 8). This means that the hemolysate was increasing constriction relative to crystalloid, as intended, but it was also preventing crystalloid from accumulating, holding the magnitude of total constriction to a greater level. This had not happened in albumin studies because the 25% albumin increased the oncotic pressure of the plasma, allowing both stressed and total volume to increase simultaneously (i.e., vessels were more relaxed compared to the shock period, but at the same time were more stretched by fluid, improving venous return).

The overarching hypothesis included the assumption that acute decompensations are the result of a sudden decrease in sympathetic output signaling (as opposed to the decreased response to that signal by blood vessels that likely underlies slow decompensations). Conditions which cause the sympathetic system to increase signaling, such as low blood pressure and ischemia, likely increase the incidence of catastrophic signal failure. By not allowing any resuscitation fluid to remain in the vasculature, and therefore not allowing any vessel relaxation, the Hemolysate Group was likely triggering greater sympathetic signaling than the Crystalloid Only Group, and thereby increasing the likelihood of acute decompensation.

The experiments show that increasing stressed volume will improve hemodynamics, but merely increasing the proportion of stressed to unstressed volume is not enough to inhibit or prevent acute decompensation. To reduce the risk of acute decompensation, there must also be an increase in total volume to reduce the burden on the sympathetic nervous system. Therefore, Experiment 2, comparing treatment with (a) a bolus of 25% NEFA-free albumin+crystalloid (b) a bolus of 25% NEFA-free albumin+hemolysate was conducted. The dosage of NEFA-free albumin (865 mg/kg) was calculated to be about equal the amount of albumin lost as a result of hemorrhage (based on 30 ml/kg bleed, 40% hematocrit, and 48 mg/ml albumin in the plasma). This dosage is the amount of albumin to restore volume to 80% of baseline at that plasma albumin concentration (i.e., the amount of albumin administered was insufficient to replace the 12 ml/kg of lost red blood cells with plasma). Since the albumin was expected to cause rapid auto-transfusion from other compartments, there was a risk that the vasculature might relax with the incoming fluid, rather than partially resisting the expansion to increase stressed volume. Therefore, a minimum initial bolus of 3 ml/kg resuscitation fluid was administered to ensure at least some Hemolysate was present early on to bolster constriction.

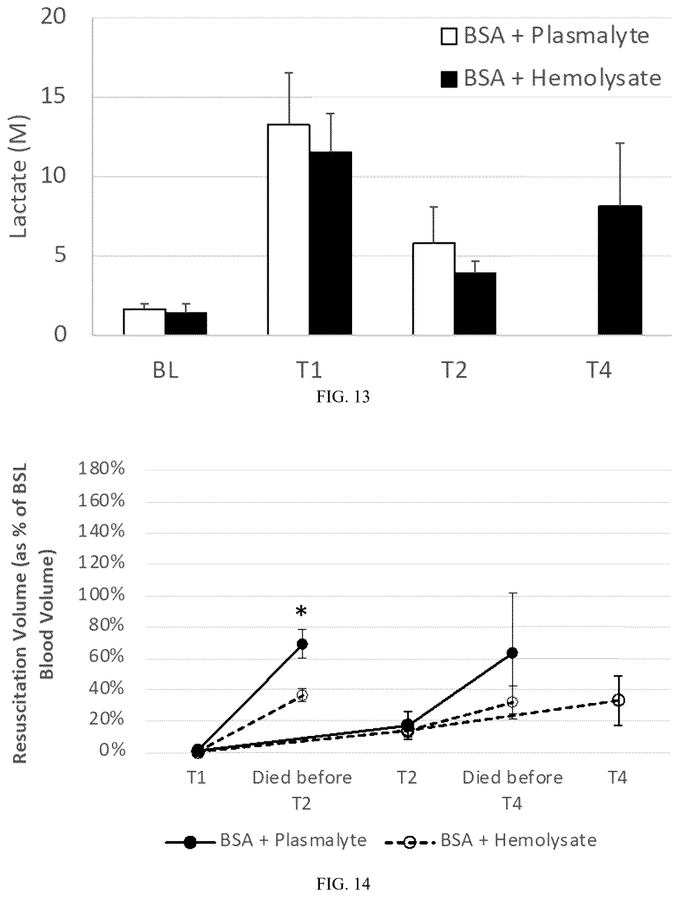

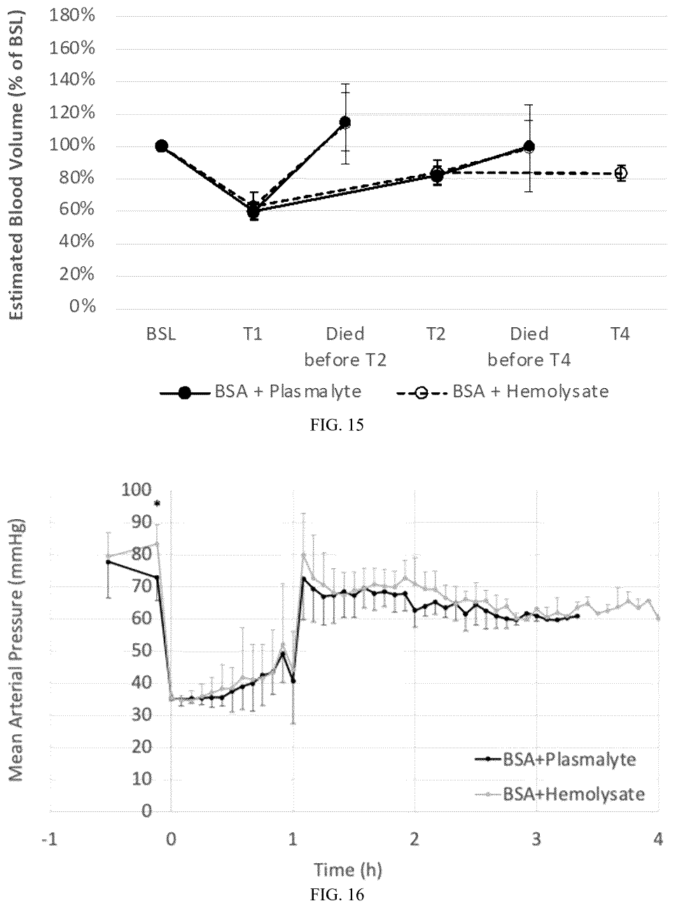

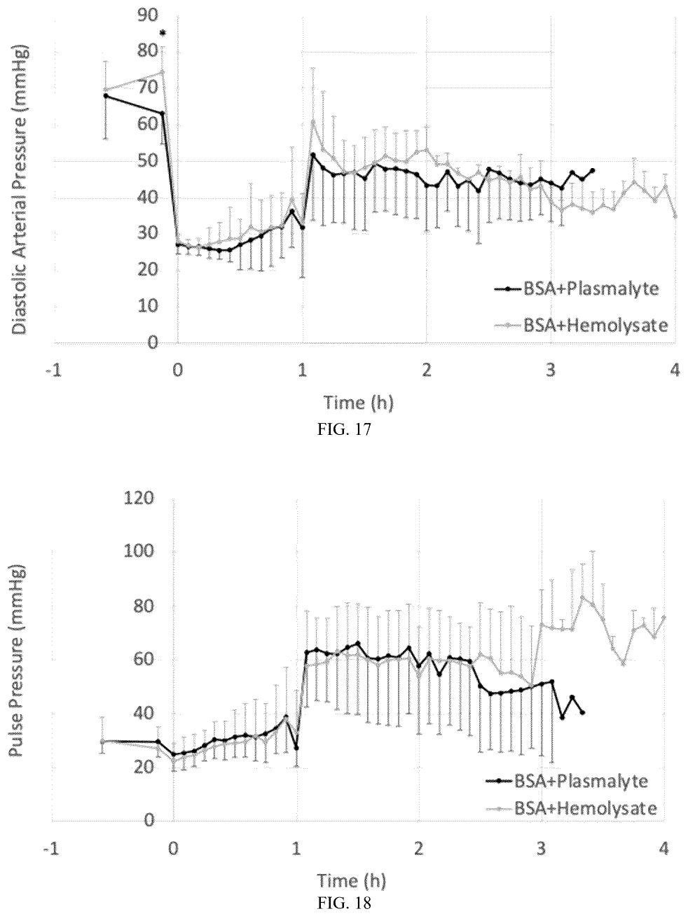

Blood pressure responded quickly with the boluses and trended higher in the NEFA-free albumin+Hemolysate Group (FIG. 16, FIG. 17, FIG. 18). Estimated blood volume in the NEFA-free albumin+Hemolysate Group increased between T1 and T2 to the goal of 80% of baseline, similar to what had been observed in the studies with BSA and oleic acid (FIG. 15). Both groups achieved the 80% baseline goal with a fraction of the volume of resuscitation fluid required in the groups without albumin, i.e., crystalloid only and hemolysate only (FIG. 14 vs. FIG. 7). The rate of resuscitation required was 0.86±0.88 ml/kg/min for the NEFA-free albumin+Crystalloid Group (about half the rate of the Crystalloid Only Group in Experiment 1) and 0.31±0.34 ml/kg/min for the NEFA-free albumin+Hemolysate Group (p=0.09 compared to NEFA-free albumin+Crystalloid Group, but p<0.0002 compared to the Crystalloid Only Group from Experiment 1). Initially, it appeared that NEFA-free albumin+Hemolysate would improve survival. Two of the first three animals in the NEFA-free albumin+Hemolysate Group survived and required less resuscitation fluid volume than was lost to hemorrhage (FIG. 14). Unfortunately, that trend did not continue, but as can be seen from FIG. 5 and FIG. 12, time of death with Hemolysate treatment went from trending worse than crystalloid treatment to trending better, when NEFA-free albumin was included. Lactate at the end of shock (T1) (prior to treatment) was extremely high (>10 mM) in all four groups (FIG. 6 and FIG. 13).

It is believed that the lack of an observable improvement in survival rates is because maximal achievable hemorrhage models were used in Experiments 1 and 2. Specifically, it is believed that severe hemorrhage for a full hour plus a tourniquet (which further increases ischemia) for two hours, pushes the stress on the sympathetic system so close to the point of failure, that acute failure will necessarily ensue (at least without a treatment that directly targets the sympathetic nervous system). Therefore, Experiments 3 and 4 were conducted to examined the effect of NEFA-free albumin+hemolysate versus NEFA-free albumin+crystalloid in a lower hemorrhage severity model (27 ml/kg blood loss). Experiment 3 used a low dosage of anesthesia (1.5% isoflurane) during the shock period to lower the severity even more, while Experiment 4 kept the dosage of anesthesia at 2% isoflurane during the shock period to achieve a severity that was intermediate between that of Experiment 3 and that of Experiments 1 and 2. This made the model conditions of Experiment 4 closest to that of the first Albumin study that showed the survival benefit of Albumin with NEFA.

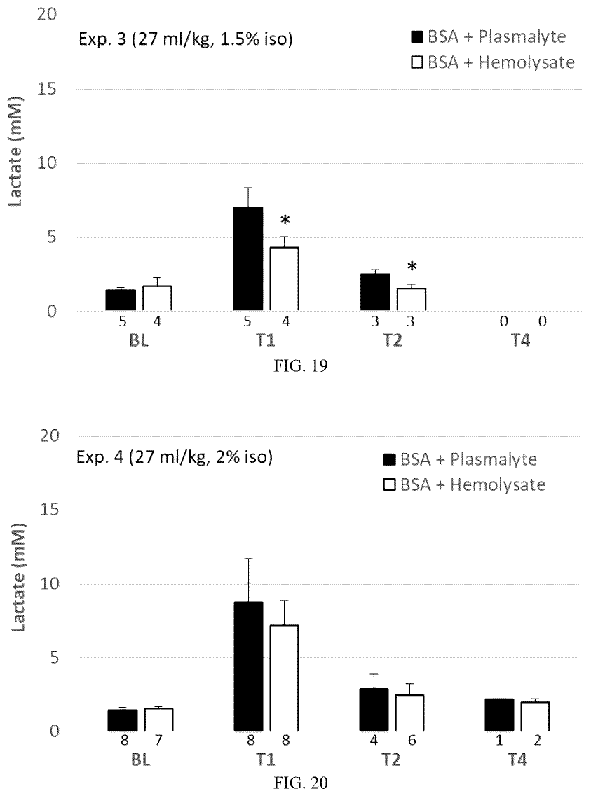

Plasma lactate levels at the end of shock (T1) (prior to treatment) in Experiment 3 were approximately half that in Experiments 1 and 2 (FIG. 19), whereas the lactate levels in Experiment 4 at T1 were approximately half way between that of Experiment 3 and that of Experiments 1 and 2 (FIG. 20). This measure is an indication of the relative severity of the models (i.e., plasma lactate indicates that Experiment 3 was the least severe, Experiment 4 had intermediate severity, and Experiments 1 and 2 were the most severe).

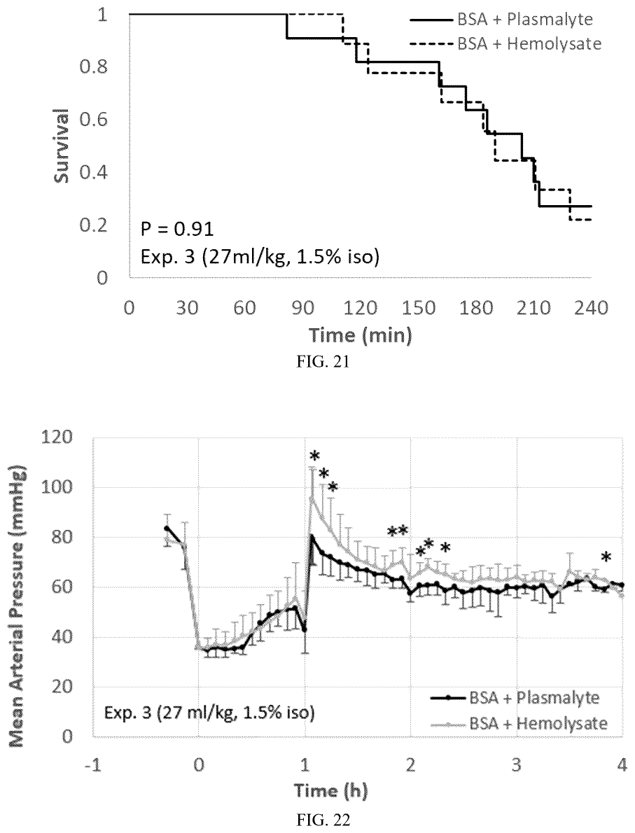

In Experiment 3, the NEFA-free albumin+Crystalloid Group had less early mortality than it had in the more severe model in Experiment 2. NEFA-free albumin+hemolysate did not extend the survival time further (FIG. 21). It did, however, significantly improve blood pressure at multiple time points (FIG. 22) and it reduced the required resuscitation rate from 0.40±0.28 ml/kg/min to 0.22±0.20 ml/kg/min (p=0.1).

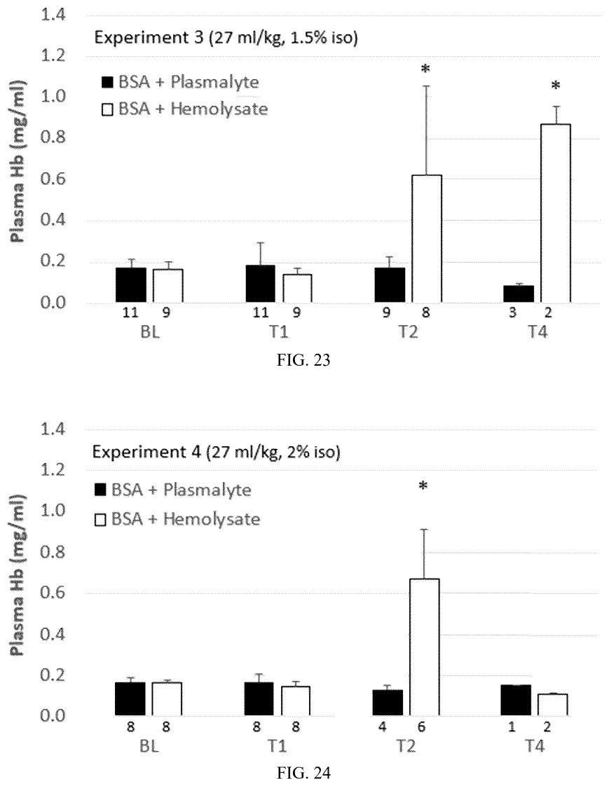

In pilot studies of the lower severity models, as more subjects were surviving to the T2 and T4 blood draws, plasma at those timepoints in groups receiving hemolysate was still red from hemoglobin, which indicates that more hemolysate than necessary was being administered. (In the NEFA-Albumin studies, accumulation of hemoglobin in the plasma was not observed.) A goal of the hemolysate component is to improve the blood response to fluid administration, so once this change in response is accomplished, pressure should be maintainable with crystalloid alone. Therefore, for Experiment 3 the maximum dosage of hemolysate was reduced from up to 3× the hemorrhaged volume, to up to 1× the hemorrhaged volume, followed by up to 2× the hemorrhage volume of crystalloid. Plasma hemoglobin levels were still elevated, however (FIG. 23). Therefore, for Experiment 4, the maximum dose of hemolysate was further reduced to a maximum of 0.25× the hemorrhage volume, followed by up to 2.75× the hemorrhage volume of crystalloid, as needed. This successfully brought plasma hemoglobin levels down to baseline by T4 (FIG. 24). Note, that this dosage corresponds to a usage of 0.24 ml/kg of packed RBCs to generate the hemolysate. For an 80 kg subject, this would translate to 19 ml of packed RBCs (or about 50 ml of whole blood), which is consistent with the previously calculated amounts of hemolysate and RBC concentrations that are believed to be therapeutically effective.

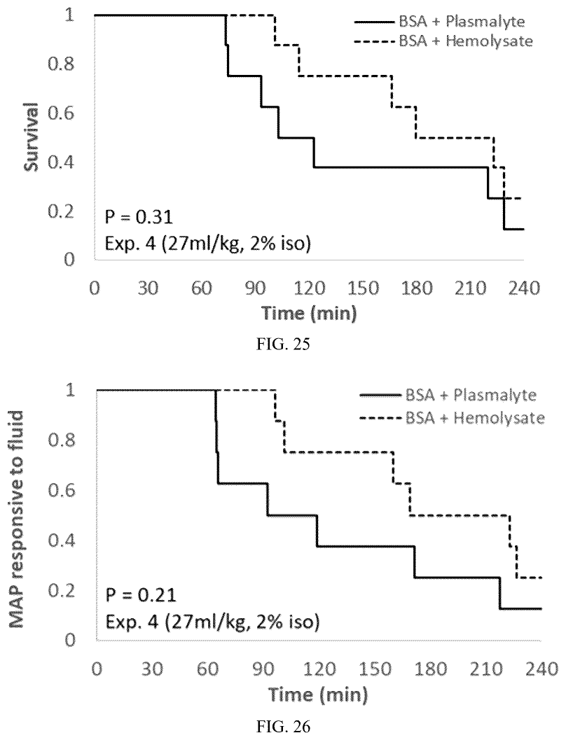

In Experiment 4, the median survival time was increased from 103 minutes in the NEFA-free albumin+Crystalloid Group to 180 minutes in the NEFA-free albumin+Hemolysate Group (p=0.3 by log-rank sum of the Kaplan-Meier curves) (FIG. 25). This improvement was even greater in the median time to shock irreversibility, when pressure stopped responding to any intervention (which is arguably an even more important measure for the pre-hospital environment than the actual time of death). Median time to shock irreversibility increased from 92 minutes in the NEFA-free albumin+Crystalloid Group to 169 minutes in the NEFA-free albumin+Hemolysate Group (p=0.2 by log-rank sum of the Kaplan-Meier curves) (FIG. 26).

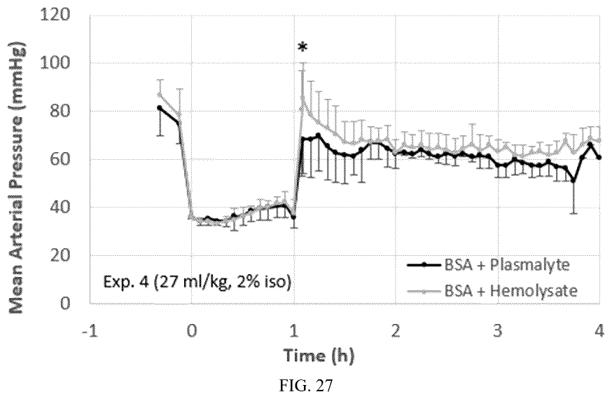

It is believed that small number of subjects (due to limited resources) employed for Experiment 4 resulted in underpowered data for detecting significance in the survival time and time to shock irreversibility. Despite the low statistical power, the improvement in blood pressure was significant immediately after the initial resuscitation boluses (FIG. 27) and the required resuscitation rate was significantly reduced 5-fold from 1.29±1.04 ml/kg/min with NEFA-free albumin+crystalloid down to 0.27±0.30 ml/kg/min with NEFA-free albumin+hemolysate (p=0.03). Based on the totality of all the experiments performed to date, one can reasonably extrapolate that treatment with NEFA-free albumin+hemolysate will improve survival times and times to shock irreversibility compared to NEFA-free albumin+crystalloid; and treatment with NEFA-free albumin+crystalloid will improve survival times and times to shock irreversibility compared to crystalloid only (no albumin). We would have also included studies in which the albumin bolus was split and administered with hemolysate at multiple time points, in which hemolysate was generated from whole blood or packed RBCs without further centrifugation, and in which the hemolysate was added to whole blood instead of crystalloid (with and without albumin bolus).

Nevertheless, the results herein indicate that treatment with NEFA-free albumin+hemolysate improves the pressure on vasculature and thereby slows the rate of decompensation. As such, resuscitation fluids comprising NEFA-free albumin+hemolysate may be used to inhibit or treat decompensation caused by severe trauma or hemorrhage and thereby provide precious additional time in which additional life-saving procedures may be performed.

### Hemolysate Preparations

In some experiments (data not shown), the hemolysate preparation included a freeze/thaw step between the addition of water to the red blood cells and centrifugation as the freeze/thaw step would increase the yield of cfHb in the hemolysate by increasing the lysis. When this hemolysate was administered to the subjects, the blood pressure tended to drop first before it eventually recovered and surpassed the pressure prior to administration. While the pressure increase was expected, the pressure drop was not. Survival was also not improved (only 1 of the 4 survived, compared to 2 of the 4 albumin+crystalloid subjects they were paired with). Furthermore, the hemolysate itself looked vastly different. The color was not as deep red (indicating less cfHb) compared to preparations without the freeze/thaw step. Additionally, the freeze/thaw step resulted in a significantly larger pellet after centrifugation (˜50% vs 1-2% by volume).

Blood smears of the supernatants of the hemolysates with and without freeze/thaw were performed. Both supernatants were transparent solutions- and neither should have had anything visible in the supernatants after 3000 g centrifugation. Though the freeze/thawed supernatant blood smear appeared empty, the supernatant of the hemolysate without freeze/thaw was surprisingly packed with what appeared to be spherical cell ghosts. This means a significant amount of the hemoglobulin in hemolysate prepared without the freeze/thaw step remains encapsulated in the lysed cell membrane.

The encapsulation explains many peculiarities. First, it explains the lack of a pellet size that is consistent with cell debris of lysed cells. The small pellet size was erroneously assumed to mean that the cell debris had simply compressed well, but the cell ghosts observed in the supernatant prove that a significant amount of the cell membranes remain in the supernatant. Second, it explains why the detrimental effects of mediators in the hemolysate (e.g., potassium) were not observed without freeze/thaw, as a significant portion likely remain encapsulated by the cell membranes. Third, it explained why a higher-than-expected concentration of hemolysate is needed to improve blood pressure, as a significant portion of hemoglobulin is also likely encapsulated by the cell membranes. Finally, it explains why some subjects, surprisingly, never needed more than an initial bolus of hemolysate to maintain a high blood pressure for hours. In theory, cfHb should have been removed from circulation in about 30 minutes or so after administration. Hemoglobulin encapsulated in cell membranes, however, would not be subjected to removal by the haptoglobin system. All of this means that removal of cell debris in the hemolysate is likely unnecessary as better results are achieved by not completely destroying the red blood cells or removing their cell ghosts. It also suggests that if, purified or recombinant hemoglobulin and/or other nitric oxide scavengers are used instead of a hemolysate preparation, they too should be encapsulated in, e.g., a lipid vesicle.

In the hemolysate experiments presented herein, hemolysate was created by combining packed RBCs with water, such that 37% of the final volume was packed RBCs (i.e., about a 37% hematocrit). This is approximately the low end of the normal range of hematocrit in humans, which corresponds to approximately 3.5×10¹² cells/L or a hemoglobin concentration of about 120 g/L, which was then centrifuged. After centrifuging, the supernatant was mixed with crystalloid in a ratio of 1:9 (supernatant to crystalloid) to make the hemolysate solution. The hemolysate solution, therefore, contained about 3.5×10¹¹ cells/L (˜12 g/L hemoglobin). In the hemolysate experiments, 3.46 ml/kg of 25% albumin (an amount based on the 30 ml/kg hemorrhage in the model) and 3 ml/kg of hemolysate were co-administered. Additional amounts of hemolysate were optionally administered. Thus, the amount of hemolysate administered ranged from about 7-28 ml/kg weight of the subject. Additional experiments using a lower hemorrhage volume (27 ml/kg), indicate that lower amounts of hemolysate are also therapeutically beneficial compared to controls. In fact, as little as the 3 ml of hemolysate per kg weight of the subject exhibited therapeutic efficacy. This corresponds to a hemolysate to albumin volumetric ratio of 0.86:1 and it is believed that the minimum relative dosage of hemolysate to albumin may be closer to 0.75:1, which corresponds to about 9 g of hemoglobin (˜2.6×10¹¹ cells) per 50-250 g of albumin. It is also believed that the upper end point of the amount of hemolysate that may be administered corresponds to a hemolysate to albumin volumetric ratio of about 8:1, which converts to about 96 g of hemoglobin (˜2.8×10¹² cells) per 50-250 g of albumin.

### Compositions

NEFA has low solubility in water. For normal transport through plasma, NEFA is bound to albumin, which then passes the NEFA to cells in a controlled fashion. NEFA is not harmful to cells while it stays bound to albumin or if it is passed to cells no faster than they are able to process it. It is only when large quantities of NEFA detach from the albumin at once and insert into cell membranes that it disrupts them. A molecule of albumin can bind multiple molecules of NEFA, but the more NEFAs bound thereto, the more easily NEFAs detach as not all the binding sites have equal affinity. Thus, administration of albumin with higher concentrations of NEFAs result in higher amounts of detached NEFAs in the circulation and thereby leads to vascular damage and leakage.

The average upper concentration limit of NEFA in the plasma of normal, healthy human subjects is about 0.5 mM. A solution of 5 wt % albumin, has about the same oncotic pressure as plasma, and is therefore considered to be a volume for volume replacement for blood loss. Therefore, 0.5 mM of NEFAs in blood is assumed to be an acceptable upper limit for NEFAs, i.e., a 0.5 mM of NEFAs in blood does not cause vascular leakage. A concentration of 0.5 mM of NEFAs in normal, healthy human adults, which is about 0.14 wt % of the amount of serum albumin in normal, healthy human adults. That is, in normal, healthy human adults, the ratio of NEFAs to albumin is about 0.14:100 by weight. Thus, while the experiments showed that albumin with NEFA of 0.02 wt % based on the weight of albumin did not cause vascular leakage, it is understood that compositions comprising an amount of NEFAs that is about 0.14 wt % of the amount of albumin in the compositions will not cause vascular leakage when administered to subjects.

As evidenced herein, administration of concentrations of albumin that are higher than the normal physiological concentration of albumin in blood, i.e., higher than about 5 wt %, pulls fluid into the vasculature from other parts of the body until the concentration of albumin normalizes. Thus, the volume of a composition containing a concentration of albumin that is higher than about 5 wt % that is needed to treat blood loss in a subject is less than the volume of blood lost. For example, the volume of a composition comprising 25 wt % albumin needed to replace blood loss in a subject is about ⅕th the volume of blood lost because the high concentration of albumin will draw fluid into the vasculature until the concentration of albumin normalizes to about 5 wt %. This means that the upper amount of NEFAs in compositions for resuscitation fluid replacement is limited by the concentration of the albumin in the concentrations. Thus, in the NEFA-free albumin compositions described herein, the total amount of NEFAs should be less than or equal to about 0.14 wt % of the albumin in the given composition. That is, in the NEFA-free albumin compositions described herein, the ratio of NEFAs to albumin by weight should be about 0-0.14:100. In some embodiments, the ratio of NEFAs to albumin by weight in the NEFA-free albumin compositions is about 0-0.07:100. In some embodiments, the ratio of NEFAs to albumin by weight in the NEFA-free albumin compositions is about 0-0.06:100. In some embodiments, the ratio of NEFAs to albumin by weight in the NEFA-free albumin compositions is about 0-0.05:100. In some embodiments, the ratio of NEFAs to albumin by weight in the NEFA-free albumin compositions is about 0-0.04:100. In some embodiments, the ratio of NEFAs to albumin by weight in the NEFA-free albumin compositions is about 0-0.03:100. In some embodiments, the ratio of NEFAs to albumin by weight in the NEFA-free albumin compositions is about 0-0.02:100. In some embodiments, the ratio of NEFAs to albumin by weight in the NEFA-free albumin compositions is about 0.02-0.14:100. In some embodiments, the ratio of NEFAs to albumin by weight in the NEFA-free albumin compositions is about 0.02-0.07:100. In some embodiments, the ratio of NEFAs to albumin by weight in the NEFA-free albumin compositions is about 0.02-0.06:100. In some embodiments, the ratio of NEFAs to albumin by weight in the NEFA-free albumin compositions is about 0.02-0.05:100. In some embodiments, the ratio of NEFAs to albumin by weight in the NEFA-free albumin compositions is about 0.02-0.04:100. In some embodiments, the ratio of NEFAs to albumin by weight in the NEFA-free albumin compositions is about 0.02-0.03:100. In some embodiments, the ratio of NEFAs to albumin by weight in the NEFA-free albumin compositions is about 0.02:100.

Thus, compositions comprising, consisting essentially of, or consisting of (a) NEFA-free albumin, (b) hemoglobulin (cfHb or “encapsulated” Hb, e.g., in the form of lysed red blood cells), c) a crystalloid, and (d) a total NEFA content of 0-0.14%, preferably 0-0.07%, more preferably 0-0.05%, and most preferably 0-0.02%, by weight of the total amount of albumin are provided herein. Albumin and NEFA-free albumin and hemoglobulin may be provided as lyophilized powders, and lysed red blood cells may be freeze dried. Thus, the NEFA-free albumin, hemoglobulin (cell free or in the form of lysed red blood cells), and crystalloid may be provided in the form of a dry admixture that is mixed with, e.g., sterile water, prior to administration. Alternatively, the compositions comprising, consisting essentially of, or consisting of (a) NEFA-free albumin, (b) hemoglobulin (cfHb or “encapsulated” Hb, e.g., in the form of lysed red blood cells), c) a crystalloid, and (d) a total NEFA content of 0-0.14%, preferably 0-0.07%, more preferably 0-0.05%, and most preferably 0-0.02%, by weight of the total amount of albumin, may themselves be aqueous formulations.

In some embodiments, the crystalloid is one or more of the following: a sodium salt, a potassium salt, a calcium salt, a magnesium salt, bicarbonate, and glucose. In some embodiments, the crystalloid is one or more of sodium chloride, sodium gluconate, sodium acetate, potassium chloride, and magnesium chloride. In some embodiments, the crystalloid is an aqueous formulation of one or more of sodium, potassium, magnesium, chloride, acetate, and gluconate. In some embodiments, the aqueous formulations (including the crystalloid itself as an aqueous formulation) comprise about 30-150 mmol/L Na⁺, about 0-15 mmol/L K⁺, about 0-1.5 mmol/L Mg²⁺, about 30-115 mmol/L Cl⁻, about 0-2 mmol/L Ca²⁺, about 0-30 mmol/L acetate, and about 0-25 mmol/L gluconate. In some embodiments, the aqueous formulations (including the crystalloid itself as an aqueous formulation) comprise about 130-140 mmol/L Na⁺, about 3-5 mmol/L K⁺, about 1-1.5 mmol/L Mg²⁺, about 95-100 mmol/L Cl⁻, about 25-30 mmol/L acetate, and about 20-25 mmol/L gluconate. In some embodiments, the crystalloid is PLASMA-LYTE 148.

In some embodiments, the compositions comprise, consist essentially of, or consist of NEFA-free albumin, hemoglobulin, and NEFAs in the following ratio based on weight 40.0-260.0:7.2-100.0:0.0-0.5, respectively. In some embodiments, the compositions comprise, consist essentially of, or consist of NEFA-free albumin, hemoglobulin, and NEFAs in the following ratio based on weight 50.0-250.0:9.0-96.0:0.0-0.4, respectively.

In some embodiments, the compositions comprise, consist essentially of, or consist of, NEFA-free albumin and per 1000 parts by weight of the albumin:
Hemoglobulin 34-2016 parts (or 1.0×10⁹-5.9×10¹⁰ lysed red blood cells per 1000 mg albumin); and
NEFAs 0.0-1.5 parts, preferably 0.0-0.8 parts.

In some embodiments, the compositions comprise, consist essentially of, or consist of, NEFA-free albumin and per 1000 parts by weight of the albumin:
Sodium Chloride 20.0-111 parts
Sodium Gluconate 19-106 parts
Sodium Acetate 14-78 parts
Potassium Chloride 1.4-8 parts
Magnesium Chloride 1.1-7 parts
Hemoglobulin 34-2016 parts (or 1.0×10⁹-5.9×10¹⁰ lysed red blood cells per 1000 mg albumin)
NEFAs 0.0-1.5 parts, preferably 0.0-0.8 parts.

In some embodiments, the compositions comprise, consist essentially of, or consist of, NEFA-free albumin and per 1000 parts by weight of the albumin (for a 25% NEFA-free albumin formulation):
Sodium Chloride 20.0-22.1 parts
Sodium Gluconate 19.1-21.1 parts
Sodium Acetate 14.0-15.5 parts
Potassium Chloride 1.4-1.6 parts
Magnesium Chloride 1.1-1.3 parts
Hemoglobulin 34-403 parts (or 1.0×10⁹-1.2×10¹⁰ lysed red blood cells per 1000 mg albumin)
NEFAs 0.0-1.5 parts, preferably 0.0-0.8 parts.

In some embodiments, the compositions comprise, consist essentially of, or consist of, NEFA-free albumin and per 1000 parts by weight of the albumin (for a 5% NEFA-free albumin formulation):
Sodium Chloride 99.9-110.5 parts
Sodium Gluconate 95.4-105.4 parts
Sodium Acetate 69.9-77.3 parts
Potassium Chloride 7.0-7.8 parts
Magnesium Chloride 5.7-6.3 parts
Hemoglobulin 171.0-2016.0 parts (or 5.0×10⁹-5.9×10¹⁰ lysed red blood cells per 1000 mg albumin)
NEFAs 0.0-1.5 parts, preferably 0.0-0.8 parts.

In some embodiments, the aqueous formulations comprise (a) about 40.0-260 g/L, preferably about 50.0-250 g/L, more preferably about 250 g/L of NEFA-free albumin; (b) about 9-96 g/L of hemoglobulin or about 2.6×10¹¹-2.8×10¹² of lysed red blood cells; and (c) a total NEFA content of about 0.14% or less, preferably about 0.07% or less, of the total amount of NEFA-free albumin.

In some embodiments, the aqueous formulations comprise, consist essentially of, or consist of:
about 40.0-260 g/L, preferably about 50.0-250 g/L, more preferably about 250 g of NEFA-free albumin;
about 30-150 mmol/L Na⁺;
about 0-15 mmol/L K⁺;
about 0-1.5 mmol/L Mg²⁺;
about 30-115 mmol/L Cl⁻;
about 0-2 mmol/L Ca²⁺;
about 0-30 mmol/L acetate;
about 0-25 mmol/L gluconate;
about 9-96 g/L of hemoglobulin or about 2.6×10¹¹-2.8×10¹² of lysed red blood cells; and
NEFAs of about 0.14% or less, preferably about 0.07% or less, of the total amount of NEFA-free albumin.

In some embodiments, the aqueous formulations comprise, consist essentially of, or consist of:
about 45.0-260 g/L, preferably about 50.0-250 g/L, more preferably about 250 g of NEFA-free albumin;
about 130-140 mmol/L Na⁺;
about 3-5 mmol/L K⁺;
about 1-1.5 mmol/L Mg²⁺;
about 95-100 mmol/L Cl⁻;
about 25-30 mmol/L acetate;
about 20-25 mmol/L gluconate;
about 9-96 g/L of hemoglobulin or about 2.6×10¹¹-2.8×10¹² of lysed red blood cells; and
NEFAs of about 0.14% or less, preferably about 0.07% or less, of the total amount of NEFA-free albumin.

The compositions may include one or more of the following: a pharmaceutically acceptable vehicle, pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions may be optimized for increased stability and efficacy using methods in the art. See, e.g., Carra et al., (2007) Vaccine 25:4149-4158.

### Treatment Methods

Provided herein are methods of treating a subject for severe trauma and/or hemorrhage, which comprise administering to the subject NEFA-free albumin in combination with hemoglobulin suspended in a crystalloid. In some embodiments, the hemoglobulin is encapsulated in a lipid vesicle or a cell membrane. In some embodiments, the hemoglobulin is provided in the form of hemolysate (i.e., lysed red blood cells). In some embodiments, the NEFA-free albumin and the hemoglobulin (e.g., hemolysate) are co-administered. In some embodiments, the amount of NEFA-free albumin administered to a subject is about 500-1000 mg/kg body weight of the subject. In some embodiments, the amount of the hemoglobulin administered to the subject is about 18-384 mg/kg body weight of the subject. In some embodiments, the amount of lysed red blood cells administered to the subject is about 5.3×10⁸-1.1×10¹⁰ cells/kg body weight of the subject.

As used herein, “co-administration” refers to the administration of at least two different agents, i.e., a first agent (e.g., NEFA-free albumin) and a second agent (e.g., hemoglobulin or hemolysate) to a subject. In some embodiments, the co-administration is concurrent. In embodiments involving concurrent co-administration, the agents may be administered as a single composition, e.g., an admixture, or as two separate compositions. In some embodiments, the first agent is administered before and/or after the administration of the second agent. Where the co-administration is sequential, the administration of the first and second agents may be separated by a period of time, e.g., minutes or hours.

In some embodiments, the compositions described herein may be administered as a single dose or as a series of several doses over a given period of minutes, hours, or days. The dosages used for treatment may increase or decrease over the course of a given treatment. Optimal dosages for a given set of conditions may be ascertained by those skilled in the art using methods in the art. For example, the timing and volume of a given fluid formulation may be adjusted as needed to maintain the subject's blood pressure in a given or desired target range.

### Kits

In some embodiments, the kits comprise (a) NEFA-free albumin (in powder or liquid form) in combination with (b) a hemoglobulin (in powder or liquid form) or lysed red blood cells. In some embodiments, the kits further comprise a crystalloid (in powder or liquid form). In some embodiments, the lysed red blood cells are freeze-dried.

In some embodiments, kits for preparing the compositions described herein are provided. In some embodiments, the kits comprise a volume of sterile water packaged together with a container for collecting a blood sample from the subject to be treated and/or mixing the blood sample with the volume of sterile water to prepare a hemolysate. In some embodiments, the kits further comprise a crystalloid (in powder or liquid form) and/or NEFA-free albumin (in powder or liquid form).

In some embodiments, kits for preparing the compositions described herein are provided. In some embodiments, the kits comprise a volume of sterile water packaged together with a container for collecting a blood sample, e.g., from a blood donor or a bag of whole blood or packed RBCs, and/or mixing the blood sample with the volume of sterile water to prepare a hemolysate. In some embodiments, the kits further comprise a crystalloid (in powder or liquid form) and/or (in powder or liquid form).

The kits may further comprise one or more intravenous delivery devices, e.g., IV needles, IV tubing, and IV bags. In some embodiments, the kits further comprise one or more syringes for, e.g., injecting a hemolysate and/or a liquid formulation of NEFA-free albumin in an IV line or IV bag.

The kits may include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

The following examples are intended to illustrate but not to limit the invention.

### EXAMPLES

In the experiments herein, PLASMA-LYTE 148 IV Infusion from Baxter Healthcare (Deerfield, IL) was employed as an exemplary crystalloid.

### Animal Procedure for the Second Albumin Study

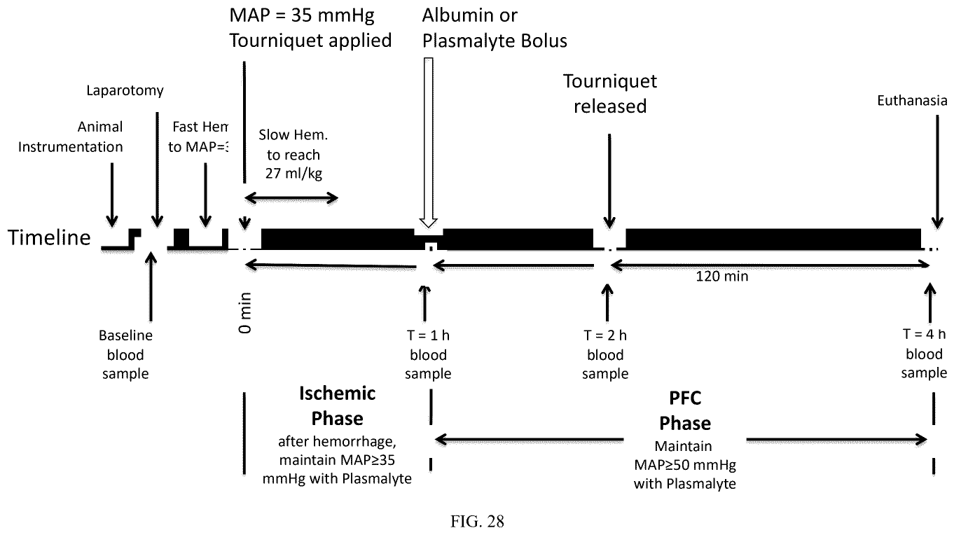

The facility's Institutional Animal Care and Use Committee (IACUC) approved all research conducted in this study. The facility where this research was conducted is fully accredited by AAALAC International. Rats were acclimated for at least 5 days after arrival at the facility prior to experimental procedures. Trauma and 27 ml/kg hemorrhagic shock models in the art were used with some modifications: 1) an extended observation period considered entirely pre-hospital to represent prolonged field care and 2) a reduction in isoflurane during hemorrhage, intended to reduce the mortality prior to resuscitation (FIG. 28). Rats were assigned into 4 treatment groups: Crystalloid (N=15), OA-saturated BSA (OA-BSA; N=17), CA-saturated BSA (CA-BSA; N=17), and pharmaceutical HSA (N=17). Adult, male Sprague Dawley rats (Charles River Laboratories, Houston, TX) between 325 and 400 g in body weight (BW) were induced with isoflurane (4%) in medical air, then maintained at 2-3% throughout instrumentation. Core temperature was maintained (36-37° C.) using a heating pad and monitored with a rectal probe (Physitemp, Clifton, NJ). The carotid artery was cannulated for continuous pressure recording, the left femoral artery for controlled hemorrhage, and the left femoral vein for blood sampling and resuscitation fluid administration. PE50 tubing for vessel cannulations was soaked overnight in heparin to reduce clotting.

Laparotomy was performed to induce trauma, then the opening was covered with plastic wrap. The right hind leg was elevated and an inflatable tourniquet (digit cuff, Hokanson, Bellevue, WA) was positioned on the leg, but not yet inflated. For the second albumin study, isoflurane was lowered to 1% immediately before start of hemorrhage. Rats were hemorrhaged (2 ml/min/kg BW) by syringe pump until MAP dropped to 35 mmHg (designated as time T=0 h). At that time, the tourniquet was inflated using a Portable Tourniquet System (Delfi Medical, Vancouver, Canada), representing first aid. The controlled hemorrhage rate was then reduced to 0.6 ml/min/kg to simulate hemorrhage not controlled by tourniquet (e.g., from another injury site or imperfect application). Blood pressure was maintained at 33-38 mmHg by adjusting the draw rate (0.2-0.8 ml/min/kg), until the total hemorrhage reached 27 ml/kg. At T=1 h, animals received a 2.95 ml/kg bolus of albumin or crystalloid, depending on assigned group, followed by permissive hypotensive resuscitation with crystalloid as needed to maintain MAP above 50 or 60 mmHg for the duration (the first albumin study maintained above 50 for the first hour and 60 for the second hour; the second albumin study maintained above 50 for the entire period; the hemolysate studies maintained above 60 for the entire period). The bolus size was chosen as the estimated dosage needed to replace the albumin lost to hemorrhage. In the second albumin study, isoflurane was also increased to 1.5% (or 2% as needed) after administration of bolus (in the first study, it had remained at 2% during the shock period, and so was not increased at this time). At T=2 h, representing the maximum recommended time before conversion to hemostatic dressing 16, the tourniquet was deflated and removed. Surviving animals were euthanized at T=4 h by intravenous administration of 0.2 ml of sodium pentobarbital euthanasia solution (Beuthanasia, Merck Animal Health, Madison, NJ), while still under anesthesia. After euthanasia, the right lung was harvested for moisture analysis (MA160, Sartorious, Bohemia, NY) and the left lung for collection of BALF (3X bronchoalveolar lavage fluid with 3 ml saline). Following the final lavage, the left lung was infused with formalin fixative for histological analysis. The liver, heart, spleen, kidney, and jejunum were also collected and fixed in formalin for histological analysis.

### Samples and Assays

Venous blood samples in heparin (0.5 ml each) were collected at baseline, as well as just prior to T=1 and 2 h, and at T=4 h. The baseline sample was included as part of the hemorrhage volume. Blood was immediately analyzed for blood gases, electrolytes, and hematocrit (ABL 800 Flex blood analyzer; Radiometer, Brea, CA). Plasma and BALF were stored at −80° C. for later analysis of protein concentration by bicinchoninic acid (BCA) assay (Sigma-Aldrich; St. Louis, MO), albumin concentration by bromocresol purple assay (Sigma-Aldrich) using reconstituted rat serum albumin (Sigma-Aldrich) for the standard, and cfHb concentration by absorbance at 405 nm in a plate reader (ELx808, BioTek; Agilent, Santa Clara, CA). Total cfHb and albumin in BALF were calculated by multiplying concentration in BALF by volume collected and normalizing by BW. Circulating blood and plasma volumes were estimated from hematocrit values using the assumption that starting blood volume is 60 ml/kg. Total protein in circulation is calculated by multiplying plasma protein concentration by estimated plasma volume.

### Albumin Treatments

25% HSA was obtained from Grifols (Albutein 25%; Los Angeles, CA). The OA- and CA-saturated BSA solutions were made fresh every two days by adding 175 μl of 2M OA or CA in ethanol to 10 ml of 250 mg/ml NEFA-free BSA in crystalloid at 37° C. The solution was then filtered through 2 glass fiber syringe filters (Pall Life Sciences, Port Washington, NY) in series to remove the excess portion of NEFA that did not bind to albumin. The hydrophobic glass fiber surfaces are capable of removing “unbound” NEFA but not NEFA bound to albumin. The OA- and CA-saturated BSA solutions were kept at 37° C. until use in the animal to avoid potential precipitation of the NEFA.

### Histological Analysis and Scoring

A blinded histological analysis of hematoxylin and eosin-stained sections was performed and graded according to their primary observed pathologies. Only the lungs displayed clinically relevant histological damage and only perivascular edema was observed there. Scoring for perivascular edema was 0 for none, 1 for Mild (perivascular space expanded up to 2× normal), 2 for Moderate (perivascular space expanded 2-5× normal), and 3 for Marked (perivascular space expanded more than 5× normal).

### Statistics

Results are described as Mean±Standard Deviation or as Median (Interquartile Range). The three Albumin Groups were first compared to each other by ANOVA or Kruskal-Wallis followed by Student's t-test or Wilcoxon Rank-Sum test (significance set at 0.017 to correct for multiple comparisons). When it became clear that the Albumin Groups were near identical in effect (see below), the Albumin Groups were combined and compared to the crystalloid control by Student's t-test or Wilcoxon Rank-Sum test (significance set to 0.05). In almost all cases, quantitative comparisons between groups, not between time points, were performed. The one exception was in the measurement of protein concentration and, therefore, of total circulating protein. This exception is due to a limitation of the BCA assay. Because the same concentration of albumin from rat, bovine, and human sources gives different results by the BCA assay, the Albumin Group values post-bolus (T2 and T4) in which albumin from multiple species is combined should not be compared unless done so qualitatively. Nevertheless, the change in protein within groups from T2 to T4 reflects gain or loss of protein from circulation during that period. A paired t-test was used for the comparison.

### NEFA-Free Albumin+Hemolysate

The hemolysate was prepared as follows: Hemorrhaged blood was centrifuged at 1000 g for 10 minutes at 4° C. to obtain a volume of packed red blood cells. 555 μl of the packed red blood cells was added to 945 μl of deionized water. After 5 minutes, the mixture was centrifuged at 1000 g for 10 minutes at 4° C. The supernatant was collected and mixed with crystalloid in a 1 to 9 volumetric ratio (supernatant to crystalloid).

Experiment 1: Anesthetized (2% isoflurane) and instrumented rats (male, 300-385 g) were given a laparotomy for trauma, then isoflurane was reduced to 1% and subjects were bled via femoral artery catheter at 2 ml/kg/min to a mean arterial pressure (MAP) of 35 mmHg (T=0 h), at which time a pneumatic tourniquet was inflated around a hind-limb (to simulate treatment) and the bleed rate was reduced to 0.6 ml/kg/min. MAP was maintained at ˜35 mmHg until 30 ml/kg of blood was withdrawn (Note: This is the maximum hemorrhage consistently achievable with this model). Resuscitation began at T=1 h with either crystalloid or Hemolysate (N=10 rats per group) and was limited to 90 ml/kg (i.e., 3× the hemorrhage volume). Hemolysate was created from the shed whole blood as described in the previous paragraph. Isoflurane was increased to 2% as soon as MAP increased to 60 mmHg. At T=2 h, the tourniquet was removed. At T=4 h, surviving subjects were euthanized. Blood was sampled (0.5 ml) at baseline, and T=1, 2, and 4 h. Baseline blood volume was assumed to be 60 ml/kg and subsequent blood volumes were calculated using hematocrit. Animals that died prior to resuscitation at T=1 h were excluded from analysis.

Experiment 2: In a second set of studies (N=10 rats per group), 3.46 ml/kg of 25% NEFA-free bovine serum albumin (BSA) was given as an IV bolus at the start of resuscitation (T=1 h), followed immediately by a minimum of 3 ml/kg of crystalloid or hemolysate, then resuscitation with crystalloid or hemolysate was administered as needed to maintain MAP of 60 mmHg, up to a limit of 90 ml/kg.

Experiment 3: In a third set of studies (N=11 rats in BSA+Crystalloid Only Group and N=9 rats in BSA+Hemolysate Group), hemorrhage was reduced to 27 ml/kg. Isoflurane was reduced from 2% to 1.5% during the shock period and restored to 2% once MAP reached 60 mmHg as in Experiments 1 and 2. NEFA-free BSA was given at the start of resuscitation as in the second set of studies. Crystalloid volume in the BSA+Crystalloid Only Group was limited to 81 ml/kg. Hemolysate volume in the BSA+Hemolysate Group was limited to 27 ml/kg, followed by up to 54 ml/kg of crystalloid, if needed.

Experiment 4: In a fourth set of studies (N=8 rats per group), hemorrhage was 27 ml/kg, but isoflurane was kept at 2% throughout the shock period and resuscitation, effectively increasing severity compared to the results of Experiment 3. NEFA-free BSA was given at the start of resuscitation as in Experiments 2 and 3. Crystalloid volume in the BSA+Crystalloid Only Group was limited to 81 ml/kg. However, the Hemolysate volume in the BSA+Hemolysate Group, was further limited to 6.75 ml/kg, followed by up to 74.25 ml/kg crystalloid, if needed.

### REFERENCES

The following references are herein incorporated by reference in their entirety with the exception that, should the scope and meaning of a term conflict with a definition explicitly set forth herein, the definition explicitly set forth herein controls:
Penn A H, Schmid-Schönbein G W. Severe intestinal ischemia can trigger cardiovascular collapse and sudden death via a parasympathetic mechanism. Shock. September 2011; 36(3):251-62.
Penn A H, Dubick M A, Torres Filho I P. Fatty Acid Saturation of Albumin Used in Resuscitation Fluids Modulates Cell Damage in Shock: in vitro Results Using a Novel Technique to Measure Fatty Acid Binding Capacity. Shock. October 2017; 48(4):449-458.
Penn A H, Williams C E, Walters T J, Dubick M A, Torres Filho I P. Fatty Acid-Saturated Albumin Reduces High Mortality and Fluid Requirements in a Rat Model of Hemorrhagic Shock Plus Tourniquet and Hypotensive Resuscitation. Shock. February 2020; 53(2):179-188.
Fisher A D, April M D, Cunningham C, Schauer S G. Prehospital Vasopressor Use Is Associated with Worse Mortality in Combat Wounded. Prehosp Emerg Care. March-April 2021; 25(2):268-273.

All scientific and technical terms used in this application have meanings commonly used in the art unless otherwise specified.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient. In the context of compositions containing (a) NEFA-free albumin, (b) hemoglobulin, and c) crystalloid, “consists essentially of” means that the compositions may comprise additional ingredients so long as the additional ingredients to not adversely affect the activities of the (a) NEFA-free albumin, (b) hemoglobulin, and c) crystalloid. Importantly, because the compositions contain NEFA-free albumin, “consists essentially of” means that the additional ingredients cannot raise the NEFA content to over 0.14% by weight of the albumin present.

The phrase “comprises, consists essentially of, or consists of A” where A is a given article is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A composition which comprises (a) a blood serum albumin, (b) a hemoglobulin, and (c) a total non-esterified fatty acid (NEFA) content of 0.14 wt % or less of the total weight of the blood serum albumin.

2. The composition according to claim 1, wherein the blood serum albumin, the hemoglobulin, and the total NEFA content are present in a ratio, based on weight, of 40.0-260.0:7.2-100.0:0.0-0.5, respectively.

3. The composition according to claim 1, wherein the hemoglobulin is provided in the form of lysed red blood cells.

4. The composition according to claim 3, wherein the amount of lysed red blood cells is about 1.0×10⁹-5.9×10¹⁰ per 1000 grams of the blood serum albumin.

5. The composition according to claim 1, further comprising, per 1000 parts by weight of the blood serum albumin, one or more of the following:
Sodium Chloride 20.0-110.5 parts;
Sodium Gluconate 19.0-105.4 parts;
Sodium Acetate 14.0-77.3 parts;
Potassium Chloride 1.4-7.8 parts; and/or
Magnesium Chloride 1.1-6.3 parts.

6. The composition according to claim 1, wherein the blood serum albumin is human serum albumin and/or the hemoglobulin is human hemoglobulin.

7. A method of treating a subject for internal bleeding and/or hemorrhage, which comprises intravenously administering to the subject a blood serum albumin and a hemoglobulin, and, optionally, an amount of non-esterified fatty acids (NEFAs) that is not more than 0.14 wt % of the administered amount of the blood serum albumin.

8. The method according to claim 7, wherein the blood serum albumin and the hemoglobulin are administered concurrently or sequentially.

9. The method according to claim 7, wherein the blood serum albumin and the hemoglobulin are administered in the form of a composition which comprises the blood serum albumin, the hemoglobulin, and an NEFA total content of 0.14 wt % or less of the total weight of the blood serum albumin in the composition.

10. The method according to claim 9, wherein the blood serum albumin, the hemoglobulin, and the total NEFA content are present in the composition at a ratio, based on weight, of 40.0-260.0:7.2-100.0:0.0-0.5, respectively.

11. The method according to claim 9, wherein the hemoglobulin is provided in the form of lysed red blood cells.

12. The method according to claim 11, wherein the amount of lysed red blood cells is about 1.0×10⁹-5.9×10¹⁰ per 1000 grams of the blood serum albumin administered.

13. The method according to claim 7, which further comprises administering to the subject sodium chloride, sodium gluconate, sodium acetate, potassium chloride, and/or magnesium chloride.

14. The method according to claim 7, wherein the lysed red blood cells are prepared by mixing a volume of whole blood with a volume of sterile water at a ratio of about 1:3 to 1:1.

15. The method according to claim 7, wherein the lysed red blood cells are prepared by mixing a volume of concentrated red blood cells with a volume of sterile water at a ratio of about 2:3 to 1:1.

16. The method according to claim 7, wherein the blood serum albumin is human serum albumin and/or the hemoglobulin is human hemoglobulin.

17. A kit comprising (a) a blood serum albumin containing 0.14 wt % or less of NEFAs, and (b) a crystalloid, a hemoglobulin, and/or lysed red blood cells packaged together.

18. The kit according to claim 17, wherein the blood serum albumin is human serum albumin and/or the hemoglobulin is human hemoglobulin.
